(19) United States
(12) Patent Application Publication
(10) Pub. No.: US 2008/0200950 A1
(43) Pub. Date: Aug. 21, 2008

(54) Surgical Hook

(76) Inventor: Stephen Wohlert, Norderstedt (DE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/575,807

(22) PCT Filed: Aug. 16, 2005
(86) PCT No.: PCT/EP2005/008887

§ 371 Date: Dec. 13, 2007

(30) Foreign Application Priority Data
Oct. 1, 2004 (DE) 10 2004 047 974.7

(51) Int. Cl. A61B 17/08

(52) U.S. Cl. 606/221

(57) ABSTRACT

Extending from a point, namely the point of rotation (2), of a surgical hook (1), there are at least two curved arms (4, 5) whose free ends (A, B) each have a tip (6, 7) and which are curved in the same direction of rotation. The arms (4, 5) can be introduced into a patient's tissue by turning the hook (1) about the point of rotation (2). The hook (1) preferably comprises absorbable material, and the arms (4, 5) are preferably provided with barbs (8) which point away from the free ends (A, B) of the arms (4, 5). The hook (1) is suitable for wound closure, including in a vertical direction, and also for fixing of implants.

The invention relates to a surgical hook which can be used, for example, for closing wounds or incisions, for creating anastomoses or for fixing implants, both in open surgery and in endoscopic surgery.

Surgical clips for wound closure and fixing of implants are in widespread use and are also often employed in endoscopic surgery. These clips are generally made of metal, for example of stainless steel or titanium, in other words of plastically deformable material. For connecting wound margins, for example, the clips are inserted into the patient's tissue, and their branches (arms) are then bent in such a way that they bring the wound margins together in the desired way and cannot then slip back out again.

When metal clips of this kind are used for fixing of implants (e.g. laparoscopically for tissue repair with implant meshes in TAPP or TEP procedures), they remain permanently in the patient, and in most cases in large numbers. When used near the surface (skin), such clips are subsequently removed, but this takes up additional time and expense and places a burden on the patient.

Since it is generally desirable to leave as little foreign material as possible in a patient's body on a permanent basis, clips made of absorbable material are desirable. However, absorbable materials, for example polylactic acid, are not plastically deformable, or are plastically deformable only to a slight extent, and this restricts their use as surgical clips.

U.S. Pat. No. 4,994,073 discloses an absorbable, bow-shaped clip in which two arms or branches issue parallel to one another from the ends of a straight middle section. The ends of the arms are pointed. Such clips are suitable for fixing implants. To ensure that the clips do not slip back out again, barbs are provided. For wound closure, this kind of clip is less practical because the wound margins are not brought towards one another.

U.S. Pat. No. 5,002,562 discloses a surgical clip made of absorbable material with barbs at the ends, which is pretensioned into a bow shape and can thus be introduced into the body. After release from a clip insertion appliance, the pretensioning has the effect that the ends move towards one another, so that, for example, an incision can be closed in this way. However, the possible uses of these clips or hooks are somewhat limited.

The use of barbs on surgical suture material and clip material has long been known. Thus, straight connectors with barbs, and connectors bent at one end and with barbs, are described, for example in U.S. Pat. No. 3,716,058. U.S. Pat. No. 5,931,855 and WO 96/06565 A1, for example, disclose suture material with barbs. Many possibilities of configuring and arranging barbs in a wide variety of forms are set out in U.S. Pat. No. 3,123,077. US 2003/0041426 A1 discloses methods for forming barbs using a cutting blade.

A further disadvantage proves to be the fact that the previously known clips cannot be used along a vertical line, for example in order to clip a deep incision from the bottom upwards through several layers of tissue. Conventional clip appliers can in fact only close wounds or incisions along a horizontal line.

Moreover, conventional clips can be inserted only with difficulty into fatty tissue which is problematic in adipose patients or in body areas with a high proportion of fatty tissue. For this reason, tissue rich in fat is avoided in fixing operations, which leads to additional stresses acting on the suture line or clip line in the adjacent fascia. This is a cause of incisional hernias (Höer, Stumpf, Rosch, Klinge, Schumpelick: Prophylaxe der Narbenhernie [Prevention of incisional hernia], Chirurg 73, 2002, 881 to 887).

U.S. Pat. No. 5,007,921 discloses an S-shaped clip made of metal or absorbable material which is elastically deformable. Formed at the ends of the two arms of the “S” there is in each case a tip which points to the centre of the “S” in the projection onto the plane of the S-shape, but is angled downwards from this plane. The S-shape acts like a spring, so that the two tips move away from one another under elastic deformation and in this state can be forced into tissue areas on different sides of a wound margin. After release of the tensioning force causing the deformation, the clip draws back in slightly, so that the two wound margins are pressed against one another. This clip can only be used near the surface because it has to be pressed onto the upper face of the tissue area that is to be treated. There can be no question of using it to fix an implant.

The object of the invention is to extend the possibilities of wound closure and fixing of implants in surgery.

This object is achieved by a surgical hook according to the invention, with the features of claim 1. Advantageous embodiments of the invention are set out in the dependent claims.

The word “hook” is used here to signify that the mode of action does not depend on a plastic deformation of arms (branches) and also that the shape deviates from that of a conventional clip with two parallel branches. The invention could, however, also be called a “clip”.

The surgical hook according to the invention has at least two curved arms which extend from a point, namely the point of rotation. The free ends of the arms each have a tip. The arms are curved in the same direction of rotation, i.e. if one starts from the point of rotation and follows an arm as far as its tip, one is moving either generally along a right-hand curve or generally along a left-hand curve in all the arms. The general curvature of an arm in question here, which defines the direction of rotation, can in principle be locally superposed by an alternate curvature, e.g. with the arm extending in a wave shape.

When, for example, the hook has been brought into an incision between two tissue parts of a patient, the arms can be introduced into the tissue by means of the hook being turned around the point of rotation. This is made easier in particular if the tip areas of the arms are oriented substantially in the continuation of the end zones of the arms (i.e. comparable to the tips of curved surgical needles which are oriented in the continuation of the needle shaft) and are approximately perpendicular to the tissue parts concerned or at least do not extend at too acute an angle to them, with the result that the arms, at the start of the rotation movement, penetrate easily and substantially uniformly into the tissue parts. In the course of the rotation movement of the hook, permitted by the general curvature of the arms in the same direction of rotation, the arms draw the tissue parts together and thus close the incision. One example of the configuration of a hook according to the invention is an S-shaped hook, as will be explained in more detail below.

In preferred embodiments, the hook according to the invention comprises absorbable material, and it is preferably composed entirely of absorbable material. The hook is then absorbed during the healing process, so that no foreign material remains in the patient's body. This is especially advantageous where several hooks arranged above one another are used to close a deep incision in which the deeper-lying hooks can no longer be removed. However, it is also conceivable that the hook comprises non-absorbable material, or is even composed entirely of non-absorbable material. In these cases, the hook remains permanently in the patient's body, or it is later removed in a minor surgical intervention.

In the hook according to the invention, at least one of the arms is preferably provided with barbs which point away from the free end of the arm. The barbs do not impede, or only minimally impede, the turning-in movement of the hook, but they prevent the arm, during and after insertion into the patient's tissue, from slipping back out of said tissue. In particularly preferred embodiments, all of the arms have barbs, at least in some areas. The barbs thus permit secure anchoring of the hook in the tissue.

The barbs can be distributed on the arms either regularly or irregularly (randomly). It is also possible for the barbs not to be designed individually, but instead, for example, along a helical line or in a ring shape resulting from rings arranged one after the other and each extending about the circumference of an arm or only a partial area thereof. In these cases, the barbs are configured, for example, as more or less fin-like edges of the helical lines or rings, pointing away from the tip of the arm and preventing, or at least substantially impeding, withdrawal of the arm from the tissue. The barbs can also, for example, be arranged, as seen in the direction of rotation of the hook, on the outer side of an arm or, as seen in the direction of rotation of the hook, on the inner side of an arm.

The barbs can be distributed along the entire length of an arm on all sides. The barbs are preferably distributed at regular intervals along the outer side of the arm. They begin at the tip of the arm and extend to just shortly before the point of rotation (in order not to impede gripping of the hook with an applicator, for example a needle holder).

Numerous forms of barbs that can in principle also be used in the invention are described in U.S. Pat. No. 3,123,077.

The barbs can be produced, for example, by cutting into the material of the arms of the hook, preferably with cuts made at an acute angle, so that the depth of the cut is small and the stability of the arms is not appreciably impaired. The spacing, length and depth of the barbs are determined by the cutting tool used and by the latter's blade angle, and they can be varied across a wide range depending on the application. The cutting depth can be easily calculated from the cutting length and the cutting angle. After the cutting operation, the material of the arm has to be bent out slightly to form the barbs, in so far as it does not deflect outwards automatically. In this context, account also has to be taken of the blade angle of the cutting tool, since this also determines the geometry, above all the angle at which the barbs will project from the main body of the arm. This angle can be varied across a wide range; it is preferably between 10° and 45°. A method for cutting barbs is described in US 2003/0041426 A1.

Another possible way of forming the barbs is for the surgical hook as a whole, or the arms as a whole, to be produced as an injection-moulded part.

The hook according to the invention (or its sections provided with barbs) can be provided with an envelope of absorbable material, preferably rapidly absorbable material. Depending on the application, such an envelope facilitates the insertion of the hook, because the barbs are screened off from the patient's tissue and the hook can even be turned back counter to the direction of action of the barbs. However, it is not until absorption of the envelope that the barbs deploy their full action against withdrawal of the hook from the tissue.

As has already been mentioned, the hook according to the invention is S-shaped in a preferred embodiment. Depending on the application, the “S” can assume different forms. The point of rotation is preferably at the centre, and the two arms of the hook that are formed by the arcs of the “S” are preferably substantially of a circle shape, having the same radius and extending ca. ⅝ths to 6/8ths of a complete circle. This geometry ensures that the tips of the arms are at a favourable angle with respect to the tissue surfaces in a tissue incision when the hook is introduced into the tissue incision and its plane extends substantially parallel to the tissue surface of the patient. By turning the hook ca. 180° about the point of rotation and a rotation axis which is substantially perpendicular to the tissue surface, both arms of the hook penetrate into the tissue surfaces and draw them together, with the result that the tissue incision is closed and can unite. The S-shape also includes shapes in which the radius of curvature of the arms is not constant, as in a circle shape, but instead varies.

While preferred embodiments of the hook have two arms that can penetrate into the tissue, the hook can also have three, four or more than four arms, these arms preferably being arranged symmetrically. Such configurations are advantageous for use in the closure of trocar incisions, in order thereby to achieve a more uniform approximation with a centrically symmetric pull.

Although the aforementioned shapes of the hook are point-symmetric with respect to the point of rotation, an asymmetrical arrangement may be more advantageous for certain applications.

The dimensions of the hook are determined by the specific use. Typical lengths (that is to say, in an S-shaped hook, four times the radius of one arm for example) are between 5 mm and 50 mm; in the case of a circular cross section of an arm, the diameter of the material is, for example, between 0.1 mm and 5 mm, which also depends on the required strength and the choice of material.

In an advantageous embodiment of the invention, at least the outer areas of the arms are situated in a common plane. This is the case with the S-shaped hook considered above. Such hooks are very suitable for closure of incisions and wounds, as has been explained.

In other embodiments of the hook, at least the outer area of each arm extends in a plane that does not, however, coincide with a plane in which the outer area of another arm extends. The free ends of the arms can also lie in a plane which extends perpendicular to an axis, the rotation axis, extending through the point of rotation, said point of rotation lying outside this plane. The angle between an axis extending between the point of rotation and the free end of an arm and the rotation axis is preferably in the range of 10° to almost 90° and can, in extreme cases, also be smaller than 10°. Particularly advantageous angles lie at approximately 70°. Such hooks are thus angled and are especially suitable for fixing surgical implants, e.g planar implant meshes. During use, the point of rotation of the hook is positioned, for example, above an implant mesh or the adjoining tissue of a patient, while one arm of the hook is placed through the implant mesh and the other arm is placed on the adjoining tissue. When the hook is turned, the hook as it were “screws” the implant mesh firmly to the patient's tissue.

For uses such as the creation of anastomoses or other cases, more complex forms of the hook according to the invention are also conceivable.

The cross section of the arms of the hook can (at least in one section) be, for example, round, oval, elliptic, convex, concave, tear-shaped, triangular, trapezoid, or diamond-shaped.

Preferred cross sections are rounded, but not circularly round. Because of the bending forces that occur during application, e.g. on an arm shaped as an arc of a circle, the preferred cross-sectional shape in this direction (horizontally or perpendicularly with respect to the rotation axis) is thicker and wider than at right angles thereto (vertically or parallel with respect to the rotation axis). Thus, concave-like cross sections (e.g. as in the case of a biconcave lens) or even more complex shapes prove advantageous for optimal distribution of the loads occurring in the arm in question. The geometric moment of inertia of the cross-sectional surface of the arm is optionally adapted to the bending forces that occur in the arm when the hook is turned about the point of rotation, and this can be done by technical computation.

In advantageous embodiments of the hook according to the invention, the area around the point of rotation is provided with a grip zone which, for example, is designed in a bow shape or as a flattened formation. This area is preferably a region without barbs and is provided in a geometry permitting secure gripping of the hook with an applicator (e.g. a needle holder). In addition, it ensures correct alignment of the hook in relation to the applicator generally and to the rotation axis specifically. In the case of the angled hooks mentioned above, the angles are defined without taking into consideration a bow-shaped grip zone for example, i.e. the point of rotation is assumed to be in a tangential continuation of the arms, so that it can also lie outside the material of the hook in the area of the grip zone.

The hook according to the invention is preferably rigid. Since it is used differently than conventional clips whose branches have to be bent upon insertion, it does not have to be plastically deformable, and so the hook can easily be made from absorbable material.

The hook can also be elastically flexible, that is to say deformable in the elastic range, if appropriate with a certain hysteresis. During the application of a hook designed in this way, for example in an S-shape, the arms bend out slightly as a result of the forces that occur, and they then return to their narrower original shape, as a result of which an additional wound closure is achieved.

In the preferred embodiment, the hook is solid and has no cavities. For some applications, however, it is advantageous if the hook is partially or completely hollow or if at least one of the arms is at least partially hollow. It is possible, if so desired, to embed active substances with or without a suitable substrate in this cavity, it being possible for these active substances to exert a wide range of effects, preferably an effect that accelerates wound healing. Such substances are released either by degradation (absorption) of the hook or through specially provided openings in the wall of the hook. These openings can extend across the whole hook and be present in very large numbers. In an extreme case, the hook is composed of a kind of microporous, sponge-like body (hollow or solid) charged with the appropriate active substance. The other extreme is a kind of osmotic pump (with only one or a small number of openings).

The materials that can be considered for the hook according to the invention have already been discussed above. These are, in particular, metals, natural and synthetic polymers, and inorganic substances (glass, ceramic) preferably absorbed by biological systems. Examples of these are absorbable metals based on calcium and magnesium alloys (see, for example, DE 197 31 021 A1, U.S. Pat. No. 3,687,135, EP 0 966 979 A2), absorbable natural polymers of synthetic or animal or human origin (e.g. collagen), absorbable synthetic polymers (e.g. PLA (polylactides), PGA (polyglycolides) and their copolymers, PDO, polycaprolactone, and copolymers of these and other absorbable polymers), and absorbable inorganic substances such as absorbable glass (in certain compositions) and ceramics based on calcium phosphate (certain modifications of hydroxyapatite). Further details are given in the claims.

In the case of absorbable hooks, the times needed for complete absorption are of secondary importance; more crucial is the retaining force of the hook during the healing process, or the time until failure of the hook function. A period of functioning of a few days (for rapidly healing tissues) to several months (for slowly healing tissue with poor circulation) is preferably chosen. The breakdown in most cases takes place by the mechanism of “bulk degradation”, although surface degradation may also occur depending on the material.

The way in which the hooks according to the invention are used in a surgical intervention has already been discussed above using the examples of a flat S-shaped hook and an angled hook. The flat hook is suitable above all for wound closure (including for subcutaneous wound closure), and, in the case of a fairly long incision for example, a number of hooks can be placed along the incision. An implant mesh can be fixed, for example, with angled hooks arranged along the edge of the implant.

It is important to stress the possibilities afforded by the hooks according to the invention in closing deep wounds or incisions. The hooks can in fact be arranged above one another in several planes, i.e. “vertically”, for example to permit layered closure of an incision passing through several layers of tissue, starting from the lowermost layer of tissue. This is possible both for quite long incisions and also for punctiform injuries, e.g. trocar incisions.

The advantages of the hooks according to the invention have in part already been mentioned and are summarized below:
The shape of the hooks permits automatic approximation of the wound margins when a hook is turned about the point of rotation, which makes wound closure easier.
No bending or plastic deformation of the material is needed for the hook to function, thus making it possible to use inexpensive materials and, above all, also absorbable polymers.
The invention facilitates the routine use of absorbable hooks for wound closure or for fixing of implants, so that after a short time no foreign material remains in the patient's body.
Very rapid subcutaneous wound closure with excellent cosmetic results is permitted by the invention.
The structure of the hooks also makes it possible to introduce hooks in a vertical arrangement into tissue, which, for example after a laparotomy, reduces the tensile stress on the fascia, particularly in adipose patients. This greatly reduces the risk of herniation.
The hooks can also be used quickly and safely for fixing implants (e.g. meshes) in soft tissue.
The rapid application of the hooks means that time is saved, with all the associated advantages of this, in particular when closing long incision lines.

The invention is described below in more detail on the basis of illustrative embodiments. In the drawings:

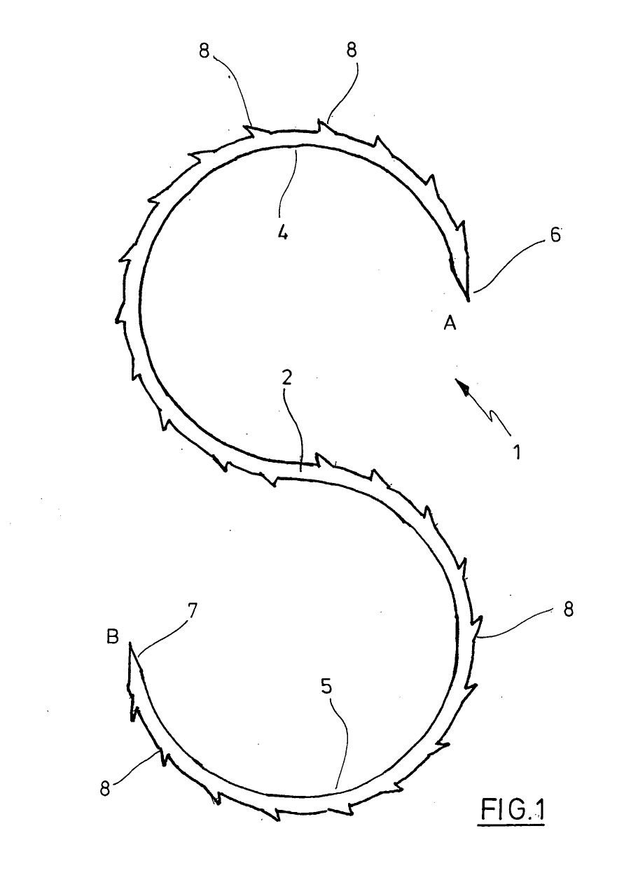

FIG. 1 shows a plan view of a first embodiment of the surgical hook according to the invention configured as a flat “S”,

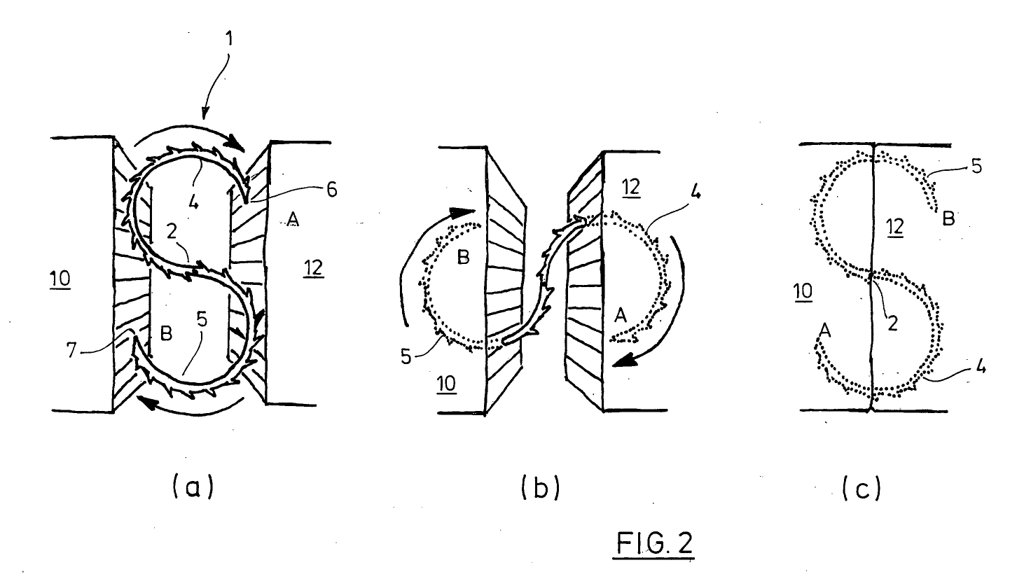

FIG. 2 shows a schematic view of several steps (parts (a) to (c)) in the application of the hook from FIG. 1,

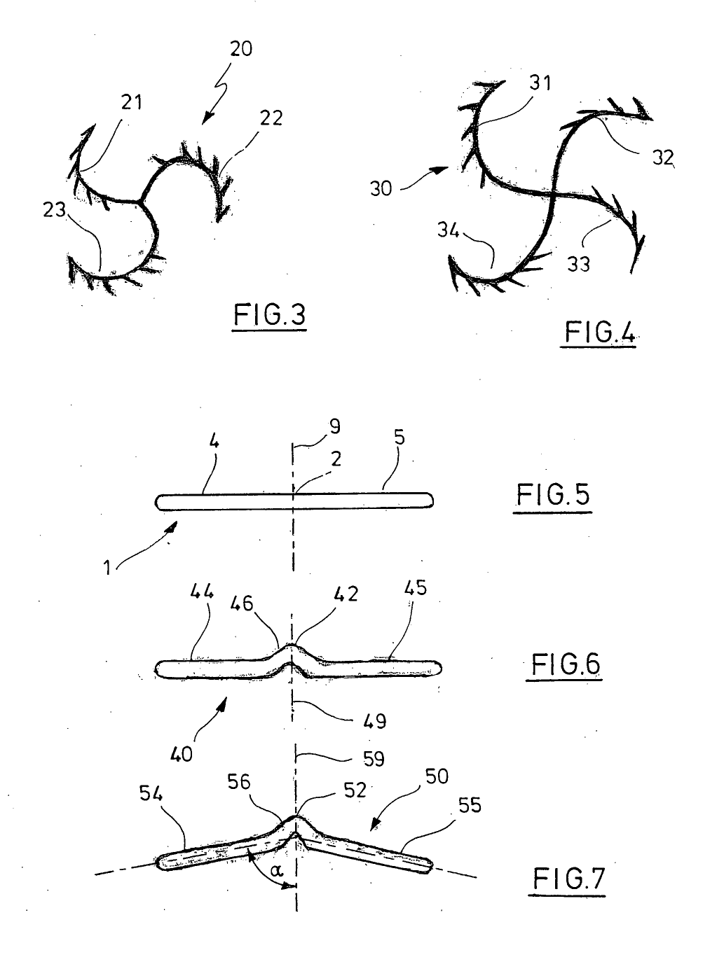

FIG. 3 shows a plan view of an embodiment of the surgical hook according to the invention with three arms,

FIG. 4 shows a plan view of an embodiment of the surgical hook according to the invention with four arms,

FIG. 5 shows a side view of the embodiment according to FIG. 1,

FIG. 6 shows a side view of a modified embodiment with a bow-shaped grip zone, and

FIG. 7 shows a side view of an angled embodiment.

An embodiment of a surgical hook 1 in the shape of the letter “S” is shown in FIG. 1. Extending from the centre point designated as the point of rotation 2, there are arms 4 and 5 which, at their respective free ends A and B, have a tip 6 and 7, respectively.

In this illustrative embodiment, the arms 4 and 5 are of a circular configuration and extend approximately 6/8ths of a complete circle. Starting from the point of rotation 2, both arms 4, 5 have the same direction of rotation. Therefore, if one goes along a respective arm 4 or 5 as far as its tip 6 or 7, one is moving along a right-hand curve or in the clockwise direction.

Barbs 8 are provided on the outer sides of the arms 4 and 5. The manner in which these barbs can be produced is described further below with reference to examples.

The hook 1 in this illustrative embodiment is flat (see also FIG. 5).

To make it easier to grip the hook 1 in the vicinity of the point of rotation 2, for example with the aid of a needle holder, which then extends substantially along a rotation axis 9 (see FIG. 5), the zone around the point of rotation 2 can have a flattened configuration (with surfaces parallel to one another extending perpendicular to the plane of the paper), while the arms 4 and 5 are made of round material, for example, or are dimensioned more strongly in the plane of the paper than perpendicular thereto.

The hook 1 can comprise non-absorbable material (e.g. metal), but is preferably made of absorbable material as explained in detail at the outset.

Referring to FIG. 2, the three steps (a), (b) and (c) show diagrammatically how the hook 1 can be used to close a wound or a surgical incision. The view is a perspective one, looking from above into the wound area with wound margins 10 and 12.

In step (a), the hook 1 is placed into the wound area with the aid of a needle holder which grips the hook 1 near the point of rotation 2 and extends substantially perpendicular to the plane of the paper. The tips 6 and 7 touch the wound margins 12 and 10, respectively, and, in the condition shown in part (a), are ready to penetrate into the tissue in the wound area.

The hook 1 is now turned in the direction of the arrow. Part (b) shows the state after a 90° turn. As will be seen, the wound margins 10, 12 have to follow the arms 4 and 5, for which reason they are moved towards one another.

Part (c) shows the state after the hook 1 has been turned through 180°, as can be seen from the free ends A and B. The wound margins 10 and 12 touch, and the wound is closed.

Tests have shown that considerable forces are needed to move the wound margins 10 and 12 apart again when the hook 1 is applied. This applies also to adipose tissue.

In the case of long incisions, several hooks 1 are placed one after the other (horizontally), and in deeper wound areas several hooks 1 are placed over one another (vertically; beginning with the lowermost hook).

FIG. 3 shows a flat hook 20 with three arms 21, 22 and 23 which are provided with barbs. FIG. 4 shows a similarly configured hook 30 with four arms 31, 32, 33 and 34. In both cases, the arms extend symmetrically. Such hooks 20 or 30 are advantageous for closing punctiform wound areas, e.g. trocar insertion points.

FIG. 5 again shows the hook 1 of the first illustrative embodiment, specifically in a side view. It will be seen that the hook 1 is flat. Moreover, the rotation axis 9 perpendicular to the plane of the paper in FIG. 1 is also shown.

FIG. 6 shows a hook 40 with a point of rotation 42 and two arms 44 and 45, said hook being of similar design to the hook 1, namely S-shaped. The zone around the point of rotation 42, however, is bent in a bow shape out from the plane of the “S”, as is made clear from the side view according to FIG. 6. In this way, a grip zone 46 is formed which makes it easier to grip the hook 40, e.g. with a needle holder, so that the hook 40 is easier to turn about a rotation axis 49. The grip zone 46 can also be used as suture abutment.

FIG. 7 shows a further embodiment of a basically S-shaped surgical hook in side view, here designated by 50. The hook 50 has a point of rotation 52, two arms 54 and 55, and a slightly less pronounced bow-shaped grip zone 56, and is turned about a rotation axis 59 upon application.

Both arms 54, 55 are kinked, specifically in such a way that the angle α between an axis along the arm 54 or 55 (more exactly along the projection of the arm 54 or 55 to the plane of the drawing) and the rotation axis 59 is about 75°. If the hook 50, in the area in which an implant overlaps a wound margin, is placed onto the implant and the wound margin, it acts like a screw, with the aid of which the implant, for example an implant mesh, can be fixed to the patient's tissue by being turned through 180°, i.e. screwed securely into place.

These examples illustrate only a small selection of the numerous possibilities afforded by the surgical hook, as has already been explained above. A few examples of the production of the surgical hook are set out below.

### EXAMPLE 1

A round steel wire (surgical steel based on a Cr—Ni alloy) measuring 0.8 mm in diameter was bent round two metal rods with a radius of 4.5 mm to give an S-shape. The wire bent in this way was then tapered conically at both ends along a distance of 2 mm (to give an equilateral triangular tip with cutting edges; the tip of the triangle points outwards in the rotation plane of the hook).

In the curved and sharpened hook, a diamond blade was then used to cut four barbs into each of the two arms of the “S”. The blade used for this has a blade angle of 45° and cuts at an angle of 6° and at a length of 0.8 mm, which, with a cutting depth of 0.084 mm, resulted in barbs having a length of 0.79 mm.

### EXAMPLE 2

A hook was first produced in an S-shape in the manner described in Example 1. After the barbs had been cut, the two arms of the hook were angled with respect to one another about the point of rotation in such a way that the above-described angle α=65°.

In an alternative, a hook was first produced in an S-shape in the manner described in Example 1. After the barbs had been cut, the two arms of the hook were bent down at the point of rotation, so that the tips of the arms came to lie about 1.5 mm below the original plane of the S-shape.

### EXAMPLE 3

Two curved surgical needles (diameter 0.3 mm, arc length ca. 8 mm) from the company called Ethicon were freed from their slide coating (desiliconized), and the reinforcement zones at the rear ends of the needles were cut off. The two remaining needles were then connected to one another by spot laser welding in such a way that a planar S-shape with an overall length of 10 mm was obtained. As has been described above, a diamond blade was then used to cut barbs on the outer side of the arc-shaped arms (blade angle: 45°; cutting angle: 6.5°; cutting length: 0.5 mm; cutting depth: 0.057 mm).

### EXAMPLE 4

Polylactic acid (poly-DL-lactide) was extruded at a temperature of 220° C. through a die with a diameter of 4 mm and then stretched to give a final diameter of 2 mm.

Following this stretching operation, the strand obtained was wound in the shape of a figure “8” around two heatable metal wires (diameter of 3 mm, distance of 2.5 mm) and fixed in this shape at a temperature of 70° C.; thermosetting generally takes place at a temperature between the glass-transition temperature and the stretching temperature.

An S-shaped hook was then cut out from the “8” in such a way that, after the tips had been ground, 5 eighths of a circle remained for each of the two arms.

Six barbs with a length of 0.99 mm and a cutting depth of 0.11 mm were then cut into the outer side of each of the two arms of the hook using a likewise heatable blade at an angle of 6.5° and a length of 1 mm.

### EXAMPLE 5

As in Example 4, polylactic acid was extruded, but the extrusion die here had the shape of a flat rectangle.

The narrow face of this rectangle when wound about the metal rods to an “8” shape was directed towards the metal rods (final dimensions of the cross section of the finished hook: 1.5 mm×2 mm).

### EXAMPLE 6

A surgical hook with barbs was produced in the desired cross section by injection moulding of polylactic acid (poly-DL-lactide) at 220° C. and subsequent cooling.

1-22. (canceled)

23. A surgical hook having first and second curved arms extending from a point to first and second free ends respectively, each of said free ends having a tip and each or said curved arms being curved in the same direction of rotation relative to the point, where the first and second curved arms can be introduced into a patient's tissue by rotating the hook about said point.

24. The hook according to claim 23, wherein the hook is comprised of an absorbable material.

25. The hook according to claim 24, wherein the absorbable material is selected from the group consisting of absorbable metals, absorbable metals based on calcium and magnesium alloys; absorbable glasses, absorbable ceramics, ceramics based on calcium phosphate, modifications of hydroxyapatite; absorbable natural polymers, absorbable synthetic polymers, polyhydroxy acids, polylactides, polyglycolides, polyhydroxy butyrates, polyhydroxy valerates, polycaprolactones, polydioxanones, synthetic and natural oligo and polyamino acids and their derivatives, in particular poly(γ-benzyl-L-glutame), pseudo-polyamino acids, proteins, especially collagens, polyphosphazenes, polyanhydrides, polyorthoesters, polyphosphates, polyphosphonates, polyalcohols, glucose polymers, in particular dextrans, dextran sulphates, chitosans, starches, hydroxyethyl starches, hydroxypropyl starches, oxygenated regenerated cellulose, hyaluronic acids, polyethers, in particular polyethyleneglycols and poly(ethyleneglycoles-co-propyleneglycoles), polycyanoacrylates, polyvinylpyrrolidones, copolymers and mixtures of such substances.

26. The hook according to claim 24, wherein the hook is comprised of a non-absorbable material selected from the group consisting of metals, stainless steel, titanium; synthetic polymers, polyalkenes, polypropylene, polyethylene, fluorinated polyolefins, polytetrafluoroethylene, polyvinylidene fluoride, polyamides, polyurethanes, polyisoprenes, polystyrenes, polysilicones, polycarbonates, polyarylether ketones, polymethacrylates, polyacrylates, aromatic polyesters, polyimides, copolymers and mixtures of such substances.

27. The hook according to claim 23, wherein at least one of the first and second curved arms is provided with a plurality of barbs that point away from the free end.

28. The hook according to claim 27, wherein the barbs have an arrangement selected from the group consisting of regularly distributed, randomly distributed, formed along a helical line, formed in a ring shape, arranged on the outer side of an arm as seen in the direction of rotation of the hook, and arranged on the inner side of an arm as seen in the direction of rotation of the hook.

29. The hook according to 27, wherein the barbs are formed by cutting into the material of at least one arm and/or by injection moulding.

30. The hook according to claim 23, wherein the hook is substantially S-shaped.

31. The hook according to claim 23, further comprising at least a third and fourth curved arms extending from the point and arranged substantially symmetrically relative to the point.

32. The hook according to claim 23 wherein the first and second arms substantially lie in a common plane.

33. The hook according to claim 23, wherein the free ends of the first and second arms lie in a plane that extends substantially perpendicular to an axis passing through a rotation axis about the point.

34. The hook according to claim 23, further comprising a grip zone substantially in proximity to the point, wherein the grip zone has a bow shape and/or a flattened formation.

35. The hook according to claim wherein at least one of the first and second arms has a cross sections selected from the group consisting of round, oval, elliptic, tear-shaped, triangular, trapezoid, and diamond-shaped.

36. The hook according to claim 23, wherein the hook is substantially rigid.

37. The hook according to claim 23, wherein the hook is substantially elastically flexible.

38. The hook according to claim 23, wherein at least one of the first and second arms is at least partially hollow.

39. The hook according to claim 23, wherein the hook is charged with at least one active substance.
